SEQUENCE LISTING (14 sequences)

SEQ ID NO: 1 - protein, 42 aa, synthetic construct
  REGION 1..42: note = Description of sequence: parent Cas9 polypeptide
KYPKLESEFVYGDYKVYDVRKMIAKSEQEIGKATAKYFFYSN

SEQ ID NO: 2 - DNA, 55 bp, synthetic construct
  misc_feature 1..55: note = Description of sequence: primer
  source 1..55: mol_type = other DNA
agctgacgtttgtactccagcgtctcatctttatgcgtcagcagagatttctgct

SEQ ID NO: 3 - DNA, 55 bp, synthetic construct
  misc_feature 1..55: note = Description of sequence: primer
  source 1..55: mol_type = other DNA
agcagaaatctctgctgacgcataaagatgagacgctggagtacaaacgtcagct

SEQ ID NO: 4 - DNA, 55 bp, synthetic construct
  misc_feature 1..55: note = Description of sequence: primer
  source 1..55: mol_type = other DNA
agctgacgtttgtactccagcgtcagttctttatgcgtcagcagagatttctgct

SEQ ID NO: 5 - DNA, 59 bp, synthetic construct
  misc_feature 1..59: note = Description of sequence: primer
  source 1..59: mol_type = other DNA
agcagaaatctctgctgacgcataaagaactgacgctggagtacaaacgtcagctctcg

SEQ ID NO: 6 - DNA, 55 bp, synthetic construct
  misc_feature 1..55: note = Description of sequence: primer
  source 1..55: mol_type = other DNA
agctgacgtttgtactccagcgtctcaagattatgcgtcagcagagatttctgct

SEQ ID NO: 7 - DNA, 59 bp, synthetic construct
  misc_feature 1..59: note = Description of sequence: primer
  source 1..59: mol_type = other DNA
agcagaaatctctgctgacgcataatcttgagacgctggagtacaaacgtcagctctcg

SEQ ID NO: 8 - DNA, 55 bp, synthetic construct
  misc_feature 1..55: note = Description of sequence: primer
  source 1..55: mol_type = other DNA
agctgacgtttgtactccagcgtctcatctaattgcgtcagcagagatttctgct

SEQ ID NO: 9 - DNA, 59 bp, synthetic construct
  misc_feature 1..59: note = Description of sequence: primer
  source 1..59: mol_type = other DNA
agcagaaatctctgctgacgcaattagatgagacgctggagtacaaacgtcagctctcg

SEQ ID NO: 10 - DNA, 55 bp, synthetic construct
  misc_feature 1..55: note = Description of sequence: primer
  source 1..55: mol_type = other DNA
agctgacgtttgtactccagcgtctcatctttaacggtcagcagagatttctgct

SEQ ID NO: 11 - DNA, 59 bp, synthetic construct
  misc_feature 1..59: note = Description of sequence: primer
  source 1..59: mol_type = other DNA
agcagaaatctctgctgaccgttaaagatgagacgctggagtacaaacgtcagctctcg

SEQ ID NO: 12 - DNA, 55 bp, synthetic construct
  misc_feature 1..55: note = Description of sequence: primer
  source 1..55: mol_type = other DNA
agctgacgtttgtactccagcgtctcatctttatgccagagcagagatttctgct

SEQ ID NO: 13 - DNA, 55 bp, synthetic construct
  misc_feature 1..55: note = Description of sequence: primer
  source 1..55: mol_type = other DNA
agcagaaatctctgctctggcataaagatgagacgctggagtacaaacgtcagct

SEQ ID NO: 14 - RNA, 20 nt, synthetic construct
  misc_feature 1..20: note = Description of sequence: Label
  source 1..20: mol_type = other RNA
gacgcataaagatgagacgc

(19) United States
(12) Patent Application Publication
(10) Pub. No.: US 2025/0043313 A1
(43) Pub. Date: Feb. 6, 2025

(54) CAS9 VARIANTS WITH IMPROVED SPECIFICITY

(71) Applicant: BOARD OF REGENTS, THE UNIVERSITY OF TEXAS SYSTEM, Austin, TX (US)

(72) Inventors: David W. TAYLOR, Austin, TX (US); Kenneth A. JOHNSON, Austin, TX (US); Jack P.K. BRAVO, Austin, TX (US); Mu-Sen LIU, Austin, TX (US); Tyler DANGERFIELD, Austin, TX (US); Grace Nicole HIBSHMAN, Austin, TX (US)

(21) Appl. No.: 18/691,246

(22) PCT Filed: Sep. 13, 2022
(86) PCT No.: PCT/US2022/076335

§ 371 Date: Mar. 12, 2024

Related U.S. Application Data
(60) Provisional application No. 63/243,481, filed on Sep. 13, 2021.
(60) Provisional application No. 63/300,443, filed on Jan. 18, 2022.

(51) Int. Cl. C12N 15/90; C12N 9/22; C12N 15/11

(52) U.S. Cl. CPC C12N 15/907; C12N 9/22; C12N 15/11; C12N 2310/20

(57) ABSTRACT

Disclosed herein are methods and compositions relating to a mutated version of Cas9. This mutation or mutations can be in the RuvC domain of Cas9. The mutated Cas9 can have decreased cleavage of mismatched target compared to a wild type Cas9.

### CROSS-REFERENCE TO RELATED APPLICATIONS

This application claims benefit of U.S. Provisional Application No. 63/243,481, filed Sep. 13, 2021 and U.S. Provisional Application No. 63/300,443, filed Jan. 18, 2022, both of which are incorporated herein by reference in their entirety.

### REFERENCE TO A SEQUENCE LISTING

The instant application contains a Sequence Listing encoded as ASCII text which was filed electronically by EFS-web and is hereby incorporated by reference in its entirety. Said ASCII text copy of the Sequence Listing, created on Sep. 13, 2022, is named “10046-447WO1_ST26.XML” and is 19,227 bytes in size.

### BACKGROUND

Genome engineering refers to the strategies and techniques for the targeted, specific modification of the genetic information (genome) of living organisms. Genome engineering is a very active field of research because of the wide range of possible applications, particularly in the areas of human health. For example, genome engineering can be used to alter (e.g., correct or knock-out) a gene carrying a harmful mutation or to explore the function of a gene. Early technologies developed to insert a transgene into a living cell were often limited by the random nature of the insertion of the new sequence into the genome. Random insertions into the genome may result in disrupting normal regulation of neighboring genes leading to severe unwanted effects. Furthermore, random integration technologies offer little reproducibility, as there is no guarantee that the sequence would be inserted at the same place in two different cells.

Clustered regularly interspaced short palindromic repeats (CRISPR) and CRISPR-associated system (CRISPR/-Cas9) is a popular tool for genome editing. However, use of CRISPR-Cas9 as a programmable genome editing tool is hindered by off-target DNA cleavage (Cong et al., 2013; Doudna, 2020; Fu et al., 2013; Jinek et al., 2013), and the underlying mechanisms by which Cas9 recognizes mismatches are poorly understood (Kim et al., 2019; Liu et al., 2020; Slaymaker and Gaudelli, 2021). Although Cas9 variants with greater discrimination against mismatches have been designed (Chen et al., 2017; Kleinstiver et al., 2016; Slaymaker et al., 2016), these suffer from significantly reduced on-target DNA cleavage rates (Kim et al., 2020; Liu et al., 2020).

While certain functions of Cas9 are linked to (but not necessarily fully determined by) their specific domains, there has been a lack of understanding of which domains correlate with changes in specificity of Cas9. For therapeutic applications of CRISPR-Cas9 to reach their full potential, it is necessary to minimize off-target DNA cleavage (Cong et al., 2013; Fu et al., 2013; Jinek et al., 2013). While a variety of “high-fidelity” Cas9 variants with improved mismatch discrimination have been developed (Chen et al., 2017; Slaymaker and Gaudelli, 2021), their enhanced specificity comes at the cost of severely reduced on-target DNA cleavage rates (Liu et al., 2020). While it has been demonstrated that mismatches induce alternative Cas9 conformations (Sternberg et al., 2015), the structures used to guide rational redesign of such variants are exclusively bound to on-target DNA, and were in inactive conformations (Anders et al., 2014; Jiang et al., 2016).

What is needed in the art are variant Cas9 molecules with increased specificity as compared to native Cas9 molecules, which are capable of retaining a high cleavage rate.

### SUMMARY

Disclosed herein is an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in the RuvC domain, and further wherein said isolated Cas9 variant or fragment has endonuclease activity.

Also disclosed is a method of performing gene editing, the method comprising contacting a target site with an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity.

Further disclosed is a method of treating a subject with a disease or disorder which is treatable with gene editing, the method comprising contacting a target site of one or more genes in need of editing within the genome of the subject with an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity, wherein said Cas9 variant or fragment edits one or more genes in a manner which effectively treats said disease or disorder.

Disclosed is a method of modifying an organism to produce a non-naturally occurring product, or a naturally occurring product in a non-naturally occurring amount, the method comprising contacting a target site of one or more genes within the genome of the organism with an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity, wherein said Cas9 edits one or more genes of interest so that the organism produces a non-naturally occurring product, or a naturally occurring product in a non-naturally occurring amount.

### BRIEF DESCRIPTION OF THE FIGURES

FIG. 1A-C shows mismatch-induced Cas9 conformational intermediates. a, Cryo-EM reconstructions of Cas9 in complex with various partially mismatched DNA substrates, determined at global resolutions ranging from 2.8-3.6 Å. Cryo-EM structures are colored according to the domain map for Cas9. Nucleotides were colored TS (green), NTS (pink) and gRNA (red). (b). Fraction of target strand (TS) DNA cleaved by Cas9 containing contiguous triple mismatches at the position and time-point used for structural determination is shown above each structure. b, Domain organization of S. Pyogenes Cas9. c, Models of Cas9 in complex with mismatched DNA substrates shown as isosurface representation. Angle between PAM-proximal and PAM-distal duplexes (θ) is shown. θ is equivalent to ˜25° for all linear conformations observed.

FIG. 2A-E shows positions 12-14 of the gRNA:TS duplex occupy a blind spot for REC3 mismatch detection. a,b, Structures of 12-14 MM at 5 min (a) and 60 min (b) in linear and kinked conformations, respectively. The position of the 12-14 MM is shown as light green and light pink for the gRNA and TS, respectively. c, Overlay and alignment of these two structures. The protein structure is largely unchanged (RMSD of <2 Å for equivalent C-alpha atoms), but the PAM-distal gRNA:TS duplex end undergoes a 30 Å conformational change, docking with REC3. d, Close-up view of positions 12-14, showing that due to the phase of the gRNA:TS duplex, REC3 makes no contacts with these base pairs. e, Schematic of interactions between REC3 and positions 9-17 of the gRNA:TS duplex. No interactions occur between Cas9 REC3 and positions 12-14 MM. Position 1 of the duplex is the first TS base that hybridizes with the gRNA spacer.

FIG. 3A-E shows linkers L1 and L2 mediate structural transition to active state. a. Overview of 18-20 MM active conformation. b,c, Detailed view of HNH (b) and RuvC (c) active sites. d, Docking of L1 linker helix against PAM-distal gRNA:TS duplex, shown as an isosurface representation. e, Interactions of L1 and L2 regions with minor groove of gRNA:TS duplex. HNH extending from L1 and L2 linkers has been removed for clarity and does not interact with this region of the gRNA:TS duplex.

FIG. 4A-G shows stabilization of distorted 18-20 MM by RuvC domain and improved fidelity of SuperFi Cas9. a, Overall structure of 18-20 MM active conformation viewed from the back. b, Schematic representation of distorted PAM-distal gRNA:TS duplex. Red circles correspond to water molecules. c & d, Close-up views of Cas9 interacting with duplex distal end. Flipped gRNA base position 2 is accommodated by stacking interactions and hydrogen bonding with RuvC tyrosine sidechains, while a network of interactions (including a water-mediated hydrogen bond) stabilizes the stretched TS configuration, allowing TS (20) to resume base-pairing with the NTS. e, Kinetics of on- and off-target (18-20 MM) cleavage by 7-D Cas9 mutant (SuperFi-Cas9Cleavage competition assay for WT Cas9 (f) and SuperFi-Cas9 (g). 25 nM of either Cas9 variant was mixed with 50 nM of each substrate and cleaved DNA product was monitored. Discrimination in favor of the on-target DNA is defined by the ratio of amplitudes for on- and off-target product formed.

FIG. 5 shows a model for Cas9 activation. During R-loop propagation (step 1), the gRNA:TS duplex adopts a linear conformation. After R-loop completion, the PAM-distal end of the linear duplex is captured by REC3 (steps 2 & 3). Mismatches in the PAM-distal region appear to prevent REC3 docking and thereby block subsequent steps of Cas9 activation. Once the kinked R-loop conformation has been formed, L1 and L2 linkers utilize the gRNA:TS duplex as a scaffold to position the HNH domain at the scissile phosphate of the TS and to position the NTS in the RuvC site (step 4), enabling Cas9 to make a double-strand break (step 5). According to this model, mutations in the RuvC loop (corresponding to SuperFi-Cas9) inhibit formation of the kinked conformation and subsequent cleavage of gRNA:TS duplex with mismatches at the PAM-distal end.

FIG. 6A-B shows kinetic basis for mismatch discrimination by Cas9. A, Schematic representation of mismatch constructs used for kinetic analysis. B, Time course of cleavage of on-target and mismatched DNA (10 nM) by Cas9. Magenta arrows correspond to time-points used to prepare cryo-EM samples. Aobs corresponds to amplitude of product formed (i.e. total cleavage). For 12-14 MM, TS cleavage is shown with larger filled circles, while NTS cleavage is given with smaller open circles. For other mismatches we only show TS cleavage. We previously reported NTS cleavage data for on-target (Gong et al., 2018) and 18-20 MM substrates (Liu et al., 2020).

FIG. 7A-C shows resolution estimates and orientation distributions of cryo-EM maps. A, Unsharpened maps colored according to local resolution. B, Gold-standard FSC curves for cryo-EM reconstructions. Resolutions were estimated at FSC=0.143. c, Euler diagrams showing orientation distributions of cryo-EM reconstructions.

FIG. 8A-D representative cryo-EM densities for 18-20 MM 1 min kinked (product) structure. A, HNH active site, showing cleaved TS. B, L1 linker docked on PAM-distal kinked gRNA:TS duplex. Two water molecules are involved within the network of interactions that stabilize the L1 helix conformation. C, RuvC active site, showing cleaved NTS, and positioning of two Mg2+ ions. D, RuvC DNA cleavage mechanism. This is a typical two-metal-ion mechanism as described by Steitz & Steitz (Steitz and Steitz, 1993) and agrees with QM/MM simulations for histidine-mediated activation (Casalino et al., 2020).

FIG. 9A-F shows structural analysis of Cas9. A, Left: Comparison of Cas9 protein only between 12-14 MM 60 min linear (color) and 12-14 MM 60 min kinked (grey) models. Right: Comparison of Cas9 protein only active conformation (18-20 MM 1 min linear, color) and kinked pre-active (12-14 MM 60 min kinked, grey) models. While there is no significant conformational change associated between transition from linear to kinked pre-active (root-mean standard deviation (RMSD) between equivalent C□ atoms of 1.904 Å), the change from kinked pre-active to active conformations is associated with a larger conformational change (4.647 Å, most of which occurs within the REC3 domain). B, Close-up view of REC3 conformational changes that occur upon activation, as viewed from one angle. REC3 moves forwards towards the kinked duplex by ˜15 Å upon activation and HNH repositioning. C, Schematic representation of Cas9: nucleic acid contacts in the context of 18-20 MM. Residues mutated in SuperFi-Cas9 are denoted by an asterisk. D, Conformations of HNH domain (green) and L1 (gold) and L2 (purple) linkers in the context of Cas9 binary complex (i.e. with gRNA, PDB 4ZT0), Cas9: gRNA complex bound to dsDNA in an inactive conformation (PDB 5F9R), and in the active Cas9 18-20 MM structure presented in this work. Upon activation, HNH is repositioned at the TS cleavage site, driven by large conformational changes in the L1 and L2 linkers. E, Comparison with the active Cas9 18-20 MM structure presented in this work and previously determined cryo-EM maps (transparent grey) of inactive (left, EMD-3276 (Jiang et al., 2016)) and active (right, EMD-0584 (Zhu et al., 2019)) Cas9 bound to on-target dsDNA. The inactive Cas9 has no density for L1 helix at the kinked distal-docked gRNA:TS site, whereas there is clear density for L1 at this site in the active Cas9 cryo-EM map. F, Mapping of residues mutated to alanine in selected high-fidelity Cas9 variants. EvoCas9 (yellow)—M495, Y515, R661, K526. Cas9-HF1 (red)—N497, R661, Q695, Q926. HypaCas9 (blue)—N692, M694, Q695, H698. Residues shared between Cas9-HF-1 and either EvoCas9 or HypaCas9 are shown as orange and purple, respectively.

FIG. 10A-B shows representative cryo-EM density for RuvC loop from two different views (a & b). Unsharpened and B-factor sharpened maps are shown for each view with the RuvC loop shown as dark magenta. Key residues involved in stabilizing this distorted conformation are labelled.

FIG. 11A-E shows RuvC loop in on-target SpCas9 structures. A, On-target inactive Cas9 bound to dsDNA (PDB 4UN3) (Anders et al., 2014). RuvC loop is missing between 1013-1029. B, On-target inactive (primed-HNH rearranged and adjacent to TS scissile phosphate) Cas9 bound to dsDNA (PDB 5F9R) (Jiang et al., 2016). RuvC loop has been built primarily as alanine ‘stub’ residues, but electron density is very poor and diffuse for this region. C, On-target inactive Cas9 bound to dsDNA (PDB 4008) (Nishimasu et al., 2015). RuvC loop is missing between 1017-1028. D, On-target active Cas9 bound to dsDNA in post-catalysis state (Zhu et al., 2019). RuvC loop is missing between 1001-1077. E, On-target active Cas9 bound to dsDNA in product state (Zhu et al., 2019). RuvC loop is missing between 1000-1075. In a-c, electron density is displayed as a grey surface, and in d & e cryo-EM density is shown as a grey surface. In all structures, missing residues are depicted as a red dashed line with the RuvC loop in b shown as magenta. Position of RuvC loop is denoted by a black dashed box in the left panel for each model.

FIG. 12A-B shows comparison of Cas9 with previous structures. A, comparison of Cas9 with a selection of previously determined structures. RMSD between equivalent C-alpha atoms is shown. B, alignment of HNH from the 18-20 MM kinked product state presented here (transparent grey) and the previously determined ‘post-catalysis’ state (PDB 600Y). The catalytically competent HNH conformation between these two structures is highly similar.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. On occasion double-stranded DNA will be referred to “duplex DNA” or “dsDNA”. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region.

“Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events; the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) o/Cell Biol 4:2253-8; Ayares et al., (1986) Proc. Natl. Acad. Sci. USA 83:5199-203; Liskay et al., (1987) Genetics 115: 161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED-5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY=0.2. Delay Divergen Seqs (%)=30, DNA Transition Weight=0.5, Protein Weight Matrix-Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, CA) using the following parameters: % identity and % similarity for a nucleotide sequence using a gap creation penalty weight of 50 and a gap length extension penalty weight of 3, and the nwsgapdna.cmp scoring matrix: % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89: 10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used) examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “promoter” is a region of DNA involved in recognition and binding of RNA polymerase and other proteins to initiate transcription. The promoter sequence consists of proximal and more distal upstream elements, the latter elements often referred to as enhancers.

An “enhancer” is a DNA sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al, (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. An RNA transcript is referred to as the mature RNA or mRNA when it is an RNA sequence derived from post-transcriptional processing of the primary transcript pre-mRNA. “Messenger RNA” or “mRNA” refers to the RNA that is without introns and that can be translated into protein by the cell.

“cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell.

“Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In another example, a target polynucleotide for cleavage by a Cas endonuclease may be of a different organism than that of the Cas endonuclease. In another example, a Cas endonuclease and guide RNA may be introduced to a target polynucleotide with an additional polynucleotide that acts as a template or donor for insertion into the target polynucleotide, wherein the additional polynucleotide is heterologous to the target polynucleotide and/or the Cas endonuclease.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1-3-2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus and includes adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas-alpha protein disclosed herein, a Cas9 protein, a Cas12a (Cpf1) protein, a Cas12b (C2c1) protein, a Cas13a (C2c2) protein, a Cas12c (C2c3) protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. The Cas-alpha endonucleases of the disclosure include those having one or more RuvC nuclease domains. A Cas protein is further defined as a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double strand break in) the target site is retained. The portion or subsequence of the Cas endonuclease can comprise a complete or partial (functional) peptide of any one of its domains.

The terms “functional variant”, “variant that is functionally equivalent” and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “Cascade” and “Cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: the most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5 ‘-5’ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be an RNA sequence, a DNA sequence, or a combination thereof (an RNA-DNA combination sequence).

The terms “functional fragment”, “fragment that is functionally equivalent” and “functionally equivalent fragment” of a guide RNA, crRNA or tracrRNA are used interchangeably herein and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, an RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, an RNA sequence, a modified DNA sequence, a modified RNA sequence (see for example EIS20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system,” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The term “transposon”, as used herein, refers to a polynucleotide (or nucleic acid segment), which may be recognized by a transposase or an integrase enzyme and which is a component of a functional nucleic acid-protein complex (e.g., a transpososome) capable of transposition. The term “transposase” as used herein refers to an enzyme, which is a component of a functional nucleic acid-protein complex capable of transposition and which mediates transposition. The transposase may comprise a single protein or comprise multiple protein sub-units. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term, “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form transpososome complex and to perform a transposition reaction.

The terms “target site”, “target sequence”, “target site sequence,” target DNA″, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition, or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

A “polynucleotide of interest” includes any nucleotide sequence encoding a protein or polypeptide that improves desirability of crops, i.e. a trait of agronomic interest. Polynucleotides of interest include but are not limited to: polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

A “complex trait locus” includes a genomic locus that has multiple transgenes genetically linked to each other.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

Efficiency is a measure of enzyme activity relative to the theoretical limit of diffusion-limited substrate binding to the enzyme (Johnson et al. 2019). Herein the term “efficiency” is used to refer to the steady-state kinetic parameter, k_{cat}/Kₘ, which is the apparent second-order rate constant for substrate binding and conversion to product. Kinetic parameters derived using direct methods as described in Gong et al. 2018, Liu et al. 2020, and Bravo et al, 2022 (herein incorporated by reference in their entirety) are implicitly given. Even though WT Cas9 catalyzes only a single enzyme turnover such that the products of the reaction remain tightly bound to the enzyme, the equations defining k_{cat}/Kₘ are still valid and will be a function of each step in the reaction cycle from substrate binding to the first largely irreversible step. For example, a mutant Cas9 molecule can have about a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in efficiency as compared to its non-mutant (native) counterpart or to another Cas9. A mutant Cas9 can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more increase in efficiency as compared to its non-mutant (native) counterpart or another Cas9.

By “specificity” is meant a function of the efficiency of reaction for a desired substrate relative to that for an undesired substrate (Johnson et al. 2019; Liu et al. 2020; Liu et al., 2019; Gong et al. 2018). Mathematically, efficiency is defined as the ratio of k_{cat}/Kₘ values to the two substrates. For Cas9, specificity is defined as (k_{cat}/Kₘ)_{on-target-DNA}/(K_{cat}/Kₘ)_{off-target-DNA}. For example, a mutant Cas9 molecule can have about a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in specificity as compared to its non-mutant (native) counterpart or to another Cas9. A mutant Cas9 can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more increase in specificity as compared to its non-mutant (native) counterpart or another Cas9.

### Systems, Compositions and Methods of Use

### General

Crystal structures have been determined for S. pyogenes Cas9 (Jinek et al., Science 343 (6176), 1247997, 2014 (“Jinek 2014”), and for S. aureus Cas9 in complex with a unimolecular guide RNA and a target DNA (Nishimasu 2014; Anders et al., Nature. 2014 Sep. 25; 513 (7519):569-73 (“Anders 2014”); and Nishimasu 2015). A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat: anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity (U.S. Pat. No. 11,098,297, herein incorporated by reference in its entirety).

In order to understand the underlying molecular mechanisms behind off-target recognition, kinetic analysis was combined with cryogenic electron microscopy (cryo-EM) to allow us to determine structural snapshots of Cas9 activation intermediates in the presence of DNA:RNA mismatches (MM). This analysis revealed unusual structural motifs that revealed a unique mechanism by which CRISPR/Cas9 stabilized the binding of an off-target DNA to form an enzyme-DNA-gRNA complex leading to DNA cleavage. This new discovery identifies specific sites on the protein where mutations would mitigate off-target DNA cleavage, leading to a more accurate variant of the CRISPR/Cas9 enzyme to meet the demands for therapeutic use.

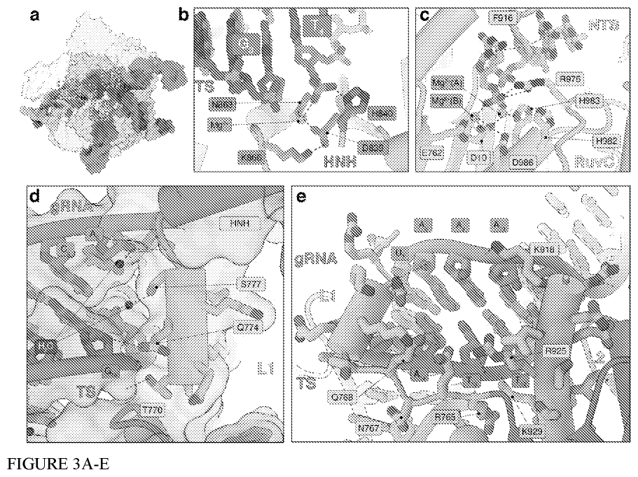

Specifically, it was observed that the DNA containing a mismatch with the guide RNA (gRNA) at positions 18-20 base pairs from the PAM recognition site (which we call 18-20 MM) contains an unusual duplex conformation at the site of the mismatch. In particular, the cytosine:cytosine mismatch at the target strand (TS) position 18 is stabilized by stacking interactions with adjacent Watson-Crick base pairs. However, the gRNA is otherwise distorted, with the gRNA position 2 flipped out by ˜180° so that the base at position I then stacks between TS positions 19 & 20. TS (19) is then contacted by Q1027:water hydrogen bond, and G20 resumes base-pairing with NTS (FIG. 3).

This unusual nucleic acid conformation is stabilized by RuvC, allowing tolerance of this mismatch. The residues within RuvC that contact and stabilize this distorted configuration are absent in previous on-target structures (Anders et al., 2014; Jiang et al., 2016; Zhu et al., 2019), indicating that they are involved only in mismatch tolerance (an essential mechanism to reduce mutational escape of Cas9 surveillance by phage) rather than on-target activation (FIG. 3).

Previously engineered variants “hyper-accurate Cas9” (HypaCas9: HypaCas9: N692A, M694A, Q695A, and H698A mutations) and “high-fidelity Cas9” (Cas9-HF1: N467A, R661A, Q695A, and Q926A mutations) have up to 100-fold slower cleavage of on-target DNA. Thus, they achieve slightly higher fidelity at the expense of a marked reduction in efficiency. The mutated residues are mainly located within the REC3 domain and make numerous interactions with the kinked duplex end. Based on these new discoveries, mutation of these residues destabilizes the kinked duplex conformation, preventing L1 docking and thereby reducing Cas9 activity for both on- and off-targets. Residues were therefore identified which are involved in mismatch tolerance that are not required for kinked duplex stabilization. These mutations are discussed herein.

In summary, Cas9 suffers from off-target cleavage of genes that closely resemble the gene intended to be targeted. This is especially true for mismatches between the guide RNA and target at positions 18-20. Disclosed herein are Cas9 variants that solve this specificity issue, allowing for precise gene editing applications. Unwanted off-target DNA cleavage by Cas9 is one of the major limitations to using the enzyme for specific gene therapy. However, the reduced gene editing efficiency of currently available high-fidelity variants means that wild-type Streptococcus pyogenes (sp) Cas9 is still by far the most widely used gene editing tool. The variants disclosed herein avoid off-target DNA cleavage, while maintaining the rapid on-target DNA cleavage rates of wild-type SpCas9.

The present disclosure encompasses, in part, the discovery of Cas9 variants that exhibit improved specificity for targeting a DNA sequence, e.g., relative to a wild-type (or native) nuclease. Provided herein are such Cas9 variants, compositions and systems that include such Cas9 variants, as well as methods of producing and methods of using such Cas9 variants, e.g., to edit one or more target nucleic acids.

### Cas9 Variants

Disclosed herein is an isolated Cas9 variant or a fragment. By “variant” or “fragment” is meant a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, or at least 99% sequence identity to a parent Cas9 polypeptide. It is noted that “parent” and “native” are referred to alternatively herein, and have the same meaning, which is the naturally occurring Cas9 on which the variant or fragment thereof is based.

Examples of naturally occurring CRISPR-Cas9s include those found in Gasiunas, G., Young, J. K., Karvelis, T. et al. A catalogue of biochemically diverse CRISPR-Cas9 orthologs. Nat Commun 11, 5512 (2020), hereby incorporated by reference in its entirety for its teaching concerning Cas9 variants. It is noted that every CRISPR-Cas9 ortholog mentioned in Gasiunas is contemplated herein.

In one example, the parent Cas9 peptide is set forth in SEQ ID NO: 1. It is noted that SEQ ID NO: 1 is based on UniProt Q99ZW2; accession codes EMD-24833 and PDB code 7S4X. SEQ ID NO: 1 is KYPKLESEFVYGDYKVYDVRKMIAKSEQEIGKATAKYFFYSN.

The isolated Cas9 variant or fragment thereof can have at least one mutation in the RuvC domain compared to the native Cas9 from which it is derived. For example, the isolated Cas9 variant or fragment can have 1, 2, 3, 4, 5, 6, 7, 8, 9, 10, 11, 12, 13, 14, 15, 16, 17, 18, 19, or 20 or more amino acid variations in the RuvC domain when compared to the native Cas9. The isolated Cas9 variant or fragment thereof can also have mutations which are not in the RuvC domain. For example, the isolated Cas9 variant or fragment thereof can have 1, 2, 3, 4, 5, 6, 7, 8, 9, 10, 11, 12, 13, 14, 15, 16, 17, 18, 19, or 20 or more amino acid variations in another area of the protein, in addition to the mutation(s) in the RuvC domain.

By way of specific example, the isolated Cas9 variant or fragment thereof, as disclosed herein, can have at least one, two, three, four, five, six, seven, eight, nine, or ten or more mutations in at least one of residues K1000, Y1001, P1002, K1003, L1004, E1005, S1006, E1007, F1008, V1009, Y1010, G1011, D1012 Y1013, K1014, V1015, Y1016, D1017, V1018, R1019, K1020, M1021, I1022, A1023, K1024, S1025, E1026, Q1027, E1028, I1029, G1030, K1031, A1032, T1033, A1034, K1035, Y1036, F1037, F1038, Y1039, S1040, or N1041 of SEQ ID NO: 1.

The isolated Cas9 variant or fragment thereof can have endonuclease activity. The isolated Cas9 variant or fragment thereof can have less, the same, or more endonuclease activity than the native Cas9 from which it is derived. For example, the isolated Cas9 variant can have a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in endonuclease activity as compared to a native Cas9. The isolated Cas9 variant or fragment thereof can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more endonuclease activity as compared to a native Cas9.

The isolated Cas9 variant or fragment thereof can have at least one improved property when compared to said parent, or native, Cas9 polypeptide. For example, the isolated Cas9 variant or fragment thereof can have improved specificity when compared to said parent Cas9 polypeptide. The term “specificity a function of the efficiency of reaction for a desired substrate relative to that for an undesired substrate. For example, the specificity of the Cas9 variant or fragment thereof can be a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in specificity as compared to its non-mutant counterpart or to another Cas9. A mutant Cas9 can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more increase in specificity as compared to its non-mutant counterpart or another Cas9. In a specific embodiment, the isolated Cas9 variant or fragment thereof does not have greater than a 10-fold decrease in specificity of as compared to said parent Cas9 polypeptide.

The Cas9 variant can have a cleavage rate which is not less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or the native, or parent, Cas9 from which it was derived. Furthermore, the Cas9 variant can have an increased cleavage rate as compared to the native Cas9. The increase can be an improvement of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more as compared to the native, or parent, Cas9.

As mentioned above, the isolated Cas9 variant or fragment thereof can have a number of different mutations or variations which can confer a number of different properties. These mutations or variations can be coupled with a mutation or variation in the RuvC domain, as disclosed herein. One of skill in the art can readily identify different variations in Cas9 that can also be incorporated into the Cas9 variants or fragments thereof disclosed herein. For example, Bak et al. (So Young Bak, Youngri Jung, Jinho Park, Keewon Sung, Hyeon-Ki Jang, Sangsu Bae, Seong Keun Kim, Quantitative assessment of engineered Cas9 variants for target specificity enhancement by single-molecule reaction pathway analysis, Nucleic Acids Research, Volume 49, Issue 19, 8-11-2021, Pages 11312-11322, herein incorporated by reference in its entirety for its teaching concerning Cas9 variants) discloses a number of these variants which are contemplated herein.

### Compositions and Kits Comprising Cas9 Variants

The Cas9 variants and fragments thereof discussed herein can be part of a composition or a kit. The composition can comprise other components which can aid in gene editing or other methods that make use of Cas9 (discussed in detail below). This is referred to herein as a “genome editing system” or “gene editing system.” For example, the composition can be a ribonucleoprotein complex, wherein said ribonucleoprotein comprises the isolated Cas9 variant or fragment thereof and a gRNA complex. The gRNA complex can comprise sgRNA, for example.

The gRNA complex can optionally comprise tracrRNA and crRNA. The ribonucleoprotein complex disclosed herein can be capable of recognizing, binding to, and optionally nicking, unwinding, or cleaving all or part of a target sequence.

Also disclosed is an expression vector encoding the isolated Cas9 variant or fragment thereof. These vectors can be part of a composition or a kit. In one embodiment, the vector can further encode a CRISPR molecule. Furthermore, the vector can encode one or more additional elements necessary to form a ribonucleoprotein complex.

Also disclosed herein are host cells, wherein a transformed host cell includes a polynucleotide that encodes for the Cas9 variant. Host cells generally refer cells that can take up exogenous materials, e.g., nucleic acids (such as DNA and RNA), polypeptides, or ribonuclear proteins. Host cells can be, e.g., single cell organisms, such as, e.g., microorganisms, or eukaryotic cells, e.g., yeast cells, mammalian cells (e.g., in culture) etc.

In some embodiments, host cells are prokaryotic cells, e.g., bacterial cells, e.g., E. coli bacteria. Bacterial cells can be Gram-negative or Gram-positive and can belong to the Bacteria (formerly called Eubacteria) domain or the Archaea (formerly called Archaebacteria) domain. Any of these types of bacteria may be suitable as host cells so long as they can be grown in a laboratory setting and can take up exogenous materials.

The host cells can be bacterial cells that are competent or made competent, e.g., in that they are able or made to be able to take up exogenous material such as genetic material. There are a variety of mechanisms by which exogenous materials such as genetic material can be introduced into host cells.

### Methods of Using Cas9

Further disclosed herein are methods of performing gene editing, the method comprising contacting a target site with a Cas9 variant as described herein. This can include the step of providing a polynucleotide encoding Cas9 to a host cell, along with other components necessary for gene editing. These methods can occur in vivo or in vitro. Those of skill in the art will understand and appreciate that there are many methods which can make use of incorporating the Cas9 variants disclosed herein into cells, both in vivo and in vitro. Incorporation of the Cas9 variants disclosed herein into cells can occur in eukaryotic cells or prokaryotic cells. When in vivo, Cas9 can be used, along with a complete gene editing system, to edit genes in an organism such as a mammal, and more specifically, such as a human. These applications are discussed below.

### In Vitro Applications

In bacteria, there are three general mechanisms, classified as transformation (uptake and incorporation of extracellular nucleic acids such as DNA), transduction (e.g., transfer of genetic material from one cell to another by a plasmid or by a virus that infects the cells, like bacteriophage), and conjugation (direct transfer of nucleic acids between two cells that are temporarily joined). Host cells into which genetic material have been introduced by transformation are generally referred to as “transformed host cells.”

In some embodiments, a polynucleotide encoding a Cas9 variant as disclosed herein is introduced into host cells by transformation. Protocols for transforming host cells are known in the art. For bacterial cells, for example, there are methods based on electroporation, methods based in lipofection, methods based on heat shock, methods based on agitation with glass beads, methods based on chemical transformation, methods based on bombardment with particles coated with exogenous material (such as DNA or RNA, etc.). One of ordinary skill in the art will be able to choose a method based on the art and/or protocols provided by manufacturers of the host cells.

Bacteriophages are viruses that infect bacteria and inject their genomes (and/or any phagemids packaged within the bacteriophage) into the cytoplasm of the bacteria. Generally, bacteriophage replicate within the bacteria, though replication-defective bacteriophage exist.

In some embodiments, a plurality of bacteriophage comprising a phagemid as described herein is incubated together with transformed host cells under conditions that allow the bacteriophage to infect the transformed host cells. The bacteriophage can be replication-competent, e.g., the bacteriophage replicate within the transformed host cells, and the replicated viral particles are released as virions in the culture medium, allowing re-infection of other host cells by bacteriophage.

Methods of the present disclosure can comprise, after the step of providing a plurality of bacteriophage comprising a phagemid that encodes a first selection agent and includes a DNA target site, a step of incubating transformed host cells (into which the polynucleotide encoding the Cas9 variant was introduced) together with a plurality of bacteriophage under culture conditions such that the plurality of bacteriophage infect the transformed host cells. Generally, these conditions are conditions in which expression of the first selection agent confers either a survival disadvantage or a survival advantage, depending on the embodiment.

In certain embodiments, the culture conditions are competitive culture conditions. “Competitive culture conditions” refers to conditions in which a population of organisms (e.g., host cells) is grown together and must compete for the same limited resources, for example, nutrients, oxygen, etc.

Host cells can be incubated in an environment in which there is no or little input of new nutrients. For example, host cells can be incubated in an environment in which there is no or little input of new oxygen, e.g., in sealed containers such as flasks.

Additionally or alternatively, host cells can be incubated in a culture medium that is well-mixed throughout the period of incubation, e.g., a shaking liquid culture. Generally, under such well-mixed conditions, the host cells have similar nutritional requirements and will be in competition for nutrients and/or oxygen (in the case of aerobic organisms) as the nutrients and/or oxygen become depleted by the growing population.

Additionally or alternatively, host cells can be incubated at an approximately constant temperature, e.g., at a temperature most suitable for the type of host cell. For example, for certain bacterial species including E. coli, host cells are typically incubated at a temperature that is around 37° C.

Host cells can be incubated in a liquid culture that is shaken. This shaking is typically vigorous enough to prevent uneven distribution of nutrients and/or settling of some host cells at the bottom of the culture. For example, host cells can be shaken at least 100 rpm (rotations per minute), at least 125 rpm, at least 150 rpm, at least 175 rpm, at least 200 rpm, at least 225 rpm, at least 250 rpm, at least 275 rpm, or at least 300 rpm. In some embodiments, host cells are shaken at between 100 rpm and 400 μm, e.g., between 200 and 350 rpm, e.g., at approximately 300 rpm.

Host cells can be incubated for a period of time before the plurality of bacteriophage is introduced into the culture. This period of time can allow, for example, the host cell population to recover from being in storage and/or to reach a particular ideal density before introduction of the plurality of bacteriophage. During this period of time before the plurality of bacteriophage is introduced, a selection pressure may be used, or it may not be used.

Culture conditions can comprise, e.g., continuous incubation of the host cells together with the bacteriophage over a period of time, e.g., at least 4 hours, at least 8 hours, at least 12 hours, or at least 16 hours. Additionally or alternatively, culture conditions can comprise continuous incubation of the host cells together with the bacteriophage until the growth of the host cells is saturated.

Culture conditions can allow continuous infection of the host cells by bacteriophage. That is, host cells are infect and re-infected continuously (if they survive) during the incubation period.

### In Vivo Applications

Disclosed herein are methods of delivering the Cas9 variants disclosed herein to subjects in need thereof. The Cas9 variant can be part of a gene editing system, as described herein. Therefore, disclosed herein is a method of modifying an organism to produce a non-naturally occurring product, or a naturally occurring product in a non-naturally occurring amount, the method comprising contacting a target site of one or more genes within the genome of the organism with an isolated Cas9 variant as described herein.

Also disclosed herein is a method of treating a subject with a disease or disorder which is treatable with gene editing, the method comprising contacting a target site of one or more genes in need of editing within the genome of the subject with an isolated Cas9 variant or a fragment thereof as described herein. This method can be used in a manner which effectively treats said disease or disorder. Contemplated herein are methods wherein the subject is an embryo.

Various diseases and/or conditions can be treated using the methods disclosed herein. For example, a single gene mutation can be treated. Various examples include, but are not limited to, cystic fibrosis, sickle cell disease, Fragile X syndrome, and muscular dystrophy.

In other embodiments, the disease and/or condition includes a dominant mutation. In various embodiments, dominance is characterized by toxic gain of function, loss of function and/or haploinsufficiency. Various examples include amyotrophic lateral sclerosis (ALS), Huntington's disease, neurofibromatosis type 1 and 2, Marfan syndrome, nonpolyposis colorectal cancer, Von Willebrand disease, among many others. In other embodiments, the disease and/or condition including a dominant mutation is retinitis pigmentosa (RP).

In other embodiments, treating the mammal for the disease and/or condition includes in vivo generation of a double stranded break (DSB) in a population of cells in the mammal. In some embodiments, a single stranded break occurs (SSB). In other embodiments, treating the disease and/or conditions includes in vivo homologous recombination (HR) of a DSB. In other embodiments, HR includes non-homologous end joining (NHEJ) introducing missense or nonsense of a protein expressed at the locus. In other embodiments, HR includes homology directed repair (HDR) introducing co-administered template DNA. In other embodiments, the co-administered template DNA is cognate to a wild-type genetic sequence. In other embodiments, the disease and/or condition includes a recessive mutation. In some embodiments, the HR results in an alteration that is an indel. In some embodiments, the HR results in an alteration causing reduced expression of the target polynucleotide sequence. In some embodiments, the HR results in an alteration that abrogates expression of a protein and/or polypeptide from the target polynucleotide sequences. In some embodiments, the alteration results in a knock out of the target polynucleotide sequence. In some embodiments, the HR results in an alteration that adjusts the target polynucleotide sequence from an undesired sequence to a desired sequence. In some embodiments, the alteration is a homozygous alteration. In some embodiments, each alteration is a homozygous alteration. In various embodiments, a quantity of stem cells, or cells differentiated from stem cells, are administered simultaneously or sequentially. Such cells can include autologous cells, including cells with alteration of a target polynucleotide sequence in the cell or cells via the described methods and compositions.

Nucleic acids encoding the various elements of a genome editing system according to the present disclosure can be administered to subjects or delivered into cells by known methods or as described herein. For example, DNA encoding an RNA-guided nuclease (e.g., an RNA-guided nuclease variant described herein) and/or encoding a gRNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA (e.g., mRNA), for instance by means of transfection or electroporation, or may be conjugated to molecules (e.g., N-acetylgalactosamine) promoting uptake by the target cells (e.g., erythrocytes, HSCs). Nucleic acid vectors may also be used.

Nucleic acid vectors can comprise one or more sequences encoding genome editing system components, such as an RNA-guided nuclease (e.g., an RNA-guided nuclease variant described herein), a gRNA and/or a donor template. A vector can also comprise a sequence encoding a signal peptide (e.g., for nuclear localization, nucleolar localization, or mitochondrial localization), associated with (e.g. inserted into, fused to) a sequence coding for a protein. As one example, a nucleic acid vectors can include a Cas9 coding sequence that includes one or more nuclear localization sequences (e.g., from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 3, and additional suitable viral vectors and their use and production are described in Cotta-Ramusino. Other viral vectors known in the art may also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which may be organic or inorganic. Nanoparticles are well known in the art, and are summarized in Cotta-Ramusino. Any suitable nanoparticle design may be used to deliver genome editing system components or nucleic acids encoding such components. For instance, organic (e.g. lipid and/or polymer) nanoparticles may be suitable for use as delivery vehicles in certain embodiments of this disclosure.

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In an embodiment, the nucleic acid molecule is delivered at the same time as one or more of the components of the Genome editing system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the genome editing system are delivered. In an embodiment, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

### Route of Administration

Genome editing systems, or cells altered or manipulated using such systems, which include the Cas9 variants disclosed herein, can be administered to subjects by any suitable mode or route, whether local or systemic. Systemic modes of administration include oral and parenteral routes. Parenteral routes include, by way of example, intravenous, intramarrow; intrarterial, intramuscular, intradermal, subcutaneous, intranasal, and intraperitoneal routes. Components administered systemically may be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In an embodiment, significantly smaller amounts of the components (compared with systemic approaches) may exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration may be provided as a periodic bolus (for example, intravenously) or as continuous infusion from an internal reservoir or from an external reservoir (for example, from an intravenous bag or implantable pump). Components may be administered locally, for example, by continuous release from a sustained release drug delivery device.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

Skilled artisans will appreciate that different components of genome editing systems can be delivered together or separately and simultaneously or nonsimultaneously. Separate and/or asynchronous delivery of genome editing system components may be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., an RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

### EXAMPLES

### Example 1: Structural Basis for Mismatch Surveillance by CRISPR/Cas9

For therapeutic applications of CRISPR-Cas9, off-target DNA cleavage must be minimized (Cong et al., 2013; Fu et al., 2013; Jinek et al., 2013). While a variety of high-fidelity Cas9 variants with improved mismatch discrimination have been developed (Chen et al., 2017; Slaymaker and Gaudelli, 2021), their enhanced specificity comes at the cost of severely reduced on-target DNA cleavage rates (Kim et al., 2020; Liu et al., 2020). While mismatches induce alternative Cas9 conformations (Singh et al., 2018; Sternberg et al., 2015), the structures used to guide rational redesign of such variants were bound to on-target DNA and in inactive conformations (Anders et al., 2014; Jiang et al., 2016). To understand the molecular mechanisms that govern off-target recognition, kinetic analysis was used to guide sample preparation for cryo-electron microscopy (cryo-EM) and structural snapshots of Cas9 pre-cleavage activation intermediates were obtained in the presence of various guide-RNA:DNA target strand (gRNA:TS) mismatches.

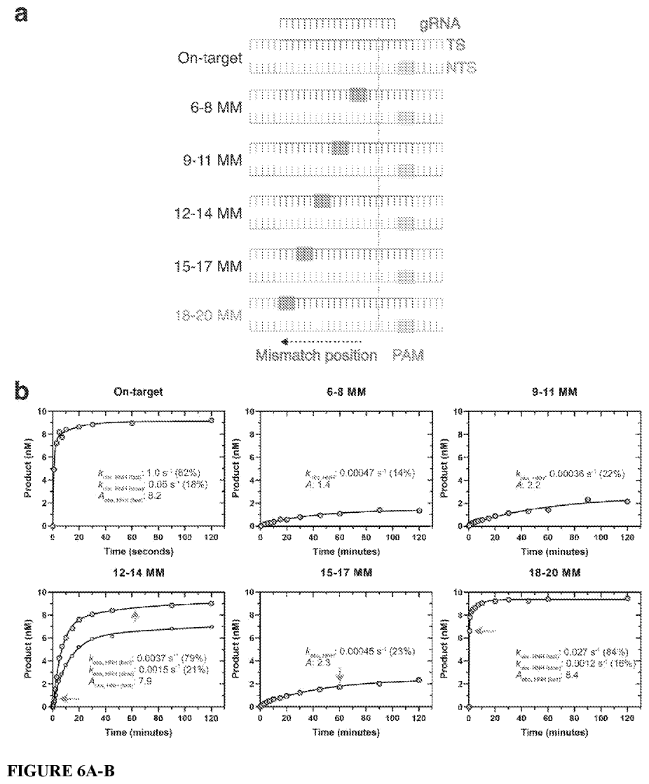

The rates of target strand (TS) cleavage by Cas9 was measured in the presence of contiguous triple nucleotide mismatches at different positions along the gRNA:TS duplex (FIG. 6a). Compared to rapid on-target cleavage (˜1.0 s⁻¹) the well-characterized PAM-distal 18-20 MM (Chen et al., 2017; Liu et al., 2020; Singh et al., 2018; Sternberg et al., 2015) (three mismatches 18-20 bp distal from the PAM) caused a ˜40-fold reduction in rate. Other mismatches (6-8 MM, 9-11 MM and 15-17 MM) resulted in a greater than 2000-fold reduction in cleavage rates with only 20% of the DNA cleaved after 2 hours of incubation (FIG. 6b).

Unexpectedly, the 12-14 MM allowed Cas9 activation but with rates ˜10-fold slower than the 18-20 MM. While Cas9 cleavage of 12-14 MM and 18-20 MM-containing DNA are both significantly slower than on-target DNA, more than 80% of either substrate was cleaved within an hour of incubation with Cas9. This time-frame for off-target cleavage poses problems for genome editing applications, which typically occur on the time-scale of days to weeks (Ran et al., 2013).

### Cryo-EM Reveals Mismatch-Induced Cas9 Intermediates

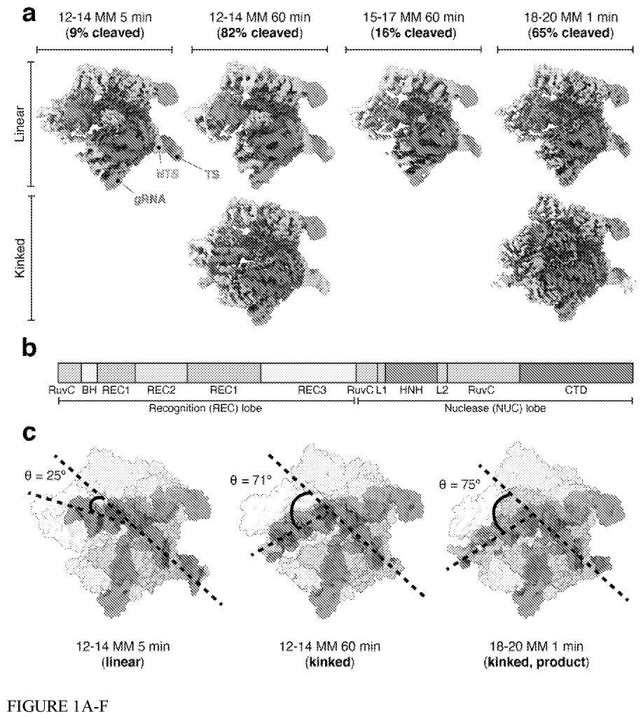

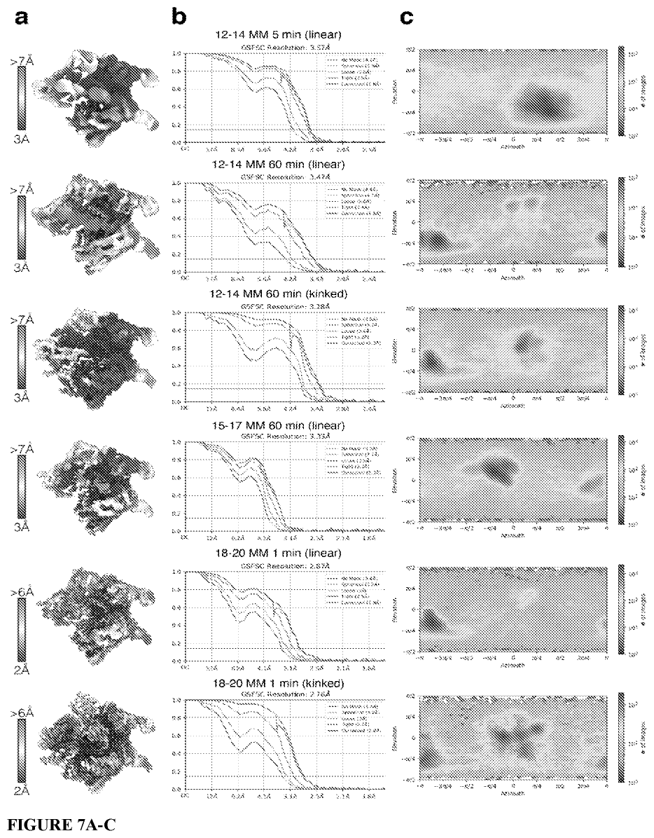

To understand the structural basis for Cas9 activation of mismatched DNA, Cas9 was vitrified with 12-14 MM DNA after a 5-minute of reaction, where only ˜10% of DNA was cleaved. A cryo-EM structure was determined at a global resolution of 3.6 Å (FIG. 1a, FIG. 7). The TS-cleaving HNH endonuclease domain was not observed, indicating conformational heterogeneity prior to activation (Dagdas et al., 2017; Zhu et al., 2019). Surprisingly, the distal end of the gRNA:TS duplex was in a linear conformation relative to the PAM-proximal DNA: DNA duplex, a state that differs from previously-determined on-target DNA-bound Cas9 structures that depict a kinked duplex (˜70°) (Jiang et al., 2016; Zhu et al., 2019). Although, this state is reminiscent of early R-loop formation intermediates (Cofsky et al., 2021).

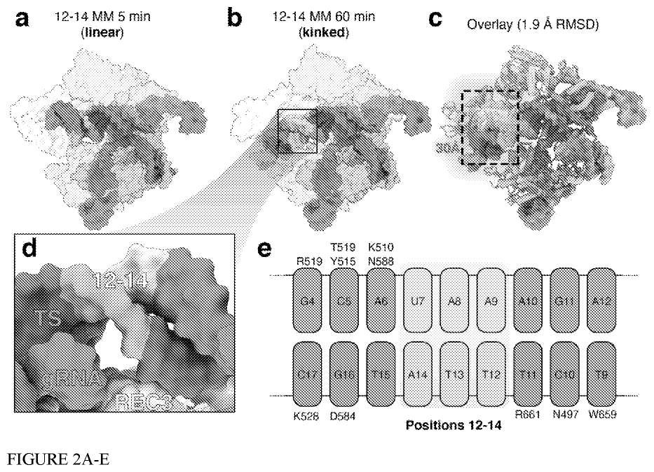

Samples of Cas9 were then vitrified with 12-14 MM DNA after a 60-minute incubation where ˜80% of the DNA was cleaved (FIG. 1b). Two distinct conformations were observed—a linear duplex conformation consistent with the 5-minute structure of 12-14 MM and the kinked duplex conformation described above (FIG. 1a,c). The Cas9 conformations in the two kinked duplex structures are identical (FIG. 2), but the PAM-distal gRNA:TS duplex end was shifted by ˜30 Å and stably docked with REC3 (FIG. 2c). It appears that the linear duplex conformation corresponds to an early intermediate of Cas9, prior to HNH rearrangement and docking to cleave the DNA (Chen et al., 2017; Zhu et al., 2019). This is supported by recent structural analyses of catalytically dead Cas9 in complex with various R-loop formation intermediates, several of which exhibit linear gRNA:TS duplex conformations that are similar to these linear duplex structures (Pacesa and Jinek, 2021).

Interestingly, positions 12-14 of the gRNA:TS make no direct contacts with the REC3 domain of Cas9 (FIG. 2). While positions 9-11 and 15-17 make significant contacts with REC3, the alignment of the gRNA:TS duplex leaves positions 12-14 without any engagement with this domain (FIG. 2d,e). Since REC3 plays a critical role in sensing PAM-distal mismatches (Chen et al., 2017), the 12-14 MM is likely able to evade mismatch discrimination by REC3 since it is positioned in a blind-spot.

It was reasoned that mismatches that prevent the PAM-distal gRNA:TS duplex from docking on REC3 would be unable to assume the kinked conformation, leading to significantly reduced DNA cleavage. To test this hypothesis, a structure of Cas9 with 15-17 MM dsDNA substrate was determined after 60 minutes of incubation with the enzyme (FIG. 1b). This mismatch inhibits cleavage by Cas9, but still permits DNA binding as measured by high-throughput profiling (Jones et al., 2021). Only the linear duplex conformation (FIG. 1a,c) was observed. These structures support a model whereby a linear duplex conformation precedes the canonical kinked duplex conformation required for activation, and mismatches that block formation of the kinked conformation escape DNA cleavage by Cas9.

### 18-20 Mismatch Supports Cas9 Conformational Activation

It was next sought how certain mismatches can evade Cas9 discrimination to allow more efficient Cas9 activation and DNA cleavage relative to other mismatches. Cas9 was examined after incubation with 18-20 MM DNA at the 1-minute time-point where ˜65% of the DNA was cleaved (FIG. 6b) to determine if this more tolerated mismatch undergoes the same structural transition as with 12-14 MM. Consistent with the fraction of product formation we observed a mixed population of particles including the linear (FIG. 1a,c) and the kinked duplex conformation. In the kinked duplex structure, HNH docked at the TS scissile phosphate, indicating the fully active conformation. This arrangement of HNH is entirely consistent with the previously observed active Cas9 conformation (Sternberg et al., 2015; Zhu et al., 2019). Importantly, these results show that the population of particles showing a linear conformation represents an early intermediate in the pathway and the kinking of the gRNA:TS duplex is linked to HNH docking.

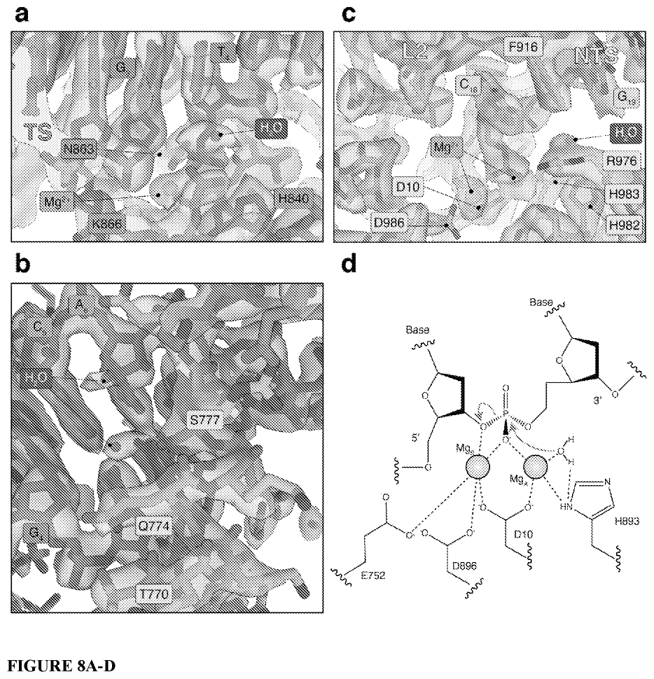

Consistent with previous studies, TS cleavage was observed between nucleotides 3 and 4 (FIG. 3, FIG. 8) and NTS cleavage at the canonical site 3 bases upstream from the PAM. The first direct observation of a RuvC active site with the non-target strand bound in the product state was observed (FIG. 3, FIG. 8). R986 is in the ‘down’ conformation, stabilizing the two magnesium ions as predicted by MD simulations (Palermo, 2019) (FIG. 3), while F916 wedges between the −2 and −3 bases via stacking interactions and positions the −3 position within the RuvC active site. These observations are in agreement with previous structural and mutagenesis studies (Jinek et al., 2014; Zhang et al., 2020). This structure suggests a histidine-mediated catalytic mechanism, consistent with two-metal-ion dependent catalysis (Steitz and Steitz, 1993) and supported by quantum-classical simulations (Casalino et al., 2020). Furthermore, the product state reveals that the two Mg2+ ions are ˜4.2 Å from each other, in agreement with the product state of the histidine-mediated mechanism (FIG. 8).

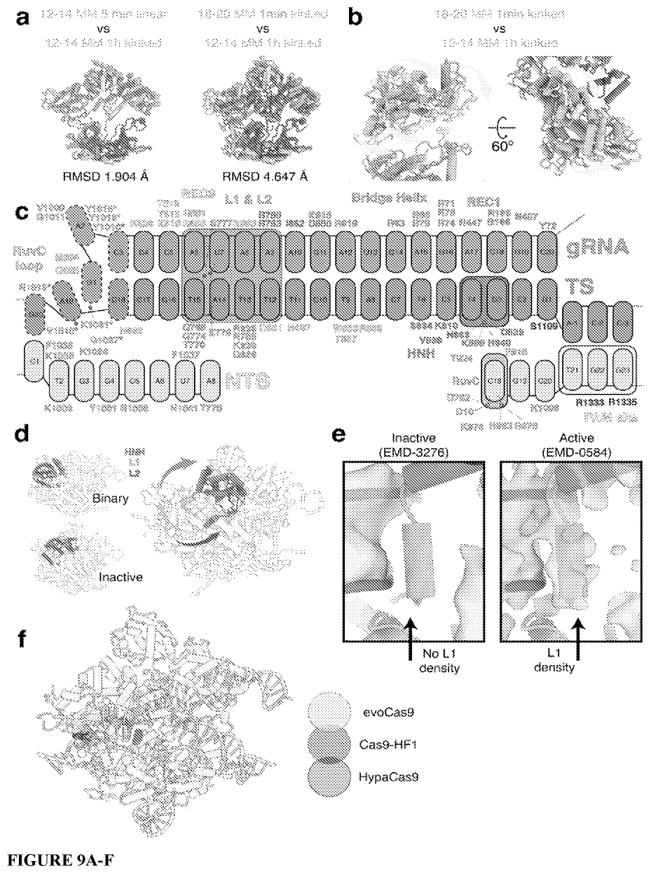

The fully active configuration requires dramatic conformational rearrangements, including a ˜140° rotation of the HNH domain from the inactive state. Furthermore, the structures reveal the molecular mechanisms underlying this rearrangement. The L1 and L2 linker domains tether HNH to the rest of Cas9 and are often missing from crystal structures, presumably due to their intrinsic flexibility. However, in the active structure, high quality density was observed for both L1 and L2. Notably, L1 helix docks against the minor groove of the PAM-distal gRNA:TS duplex and forms an extended network of interactions, including multiple water-mediated hydrogen bonds with both strands (FIG. 3). Since L1 docks on the minor groove these interactions are gRNA:TS structure-specific rather than sequence specific and can only occur when the PAM-distal duplex end is in the kinked conformation. This provides a structural basis for the observation that the kinked duplex conformation is an intermediate preceding Cas9 activation and DNA cleavage. Comparisons of this model with Cas9 structures in inactive (EMD-3276) and active (EMD-0584) conformations confirmed that L1 docking against the gRNA:TS duplex is correlated with HNH rearrangement and Cas9 activation (FIG. 9). Furthermore, the observation of L1 and L2 “locking” HNH in an active conformation is supported by the slow rate of dissociation of Cas9 from target DNA post-cleavage (Aldag et al., 2021).

Residue F916 stabilizes the non-target strand (NTS) and is within the L2 linker domain: however, within the inactive Cas9 conformation, L2 is positioned >20 Å away from the RuvC active site. L1-facilitated positioning of HNH on the TS enables relocation of L2 which in turn enables positioning of the NTS within the RuvC active site (FIG. 9). This mechanism provides a structural explanation for the observed coupling of TS and NTS cleavage where HNH docking precedes alignment of the NTS at the RuvC site for cleavage (Gong et al., 2018; Liu et al., 2020). The HNH and RuvC cleavage reactions appear to occur simultaneously because the alignment is rate-limiting.

While previous studies have noted the importance of L1 docking onto the gRNA:TS duplex for HNH repositioning (Sun et al., 2019; Zhang et al., 2020), the observation that a linear gRNA:TS duplex conformation induced by PAM-distal mismatches precludes L1 docking provides a structural explanation for why certain PAM-distal mismatched substrates are able to bind Cas9, while not triggering DNA cleavage (Jones et al., 2021).

### The 18-20 MM is Held in a Conformation to Mimic Matched DNA by Ordering of a RuvC Loop

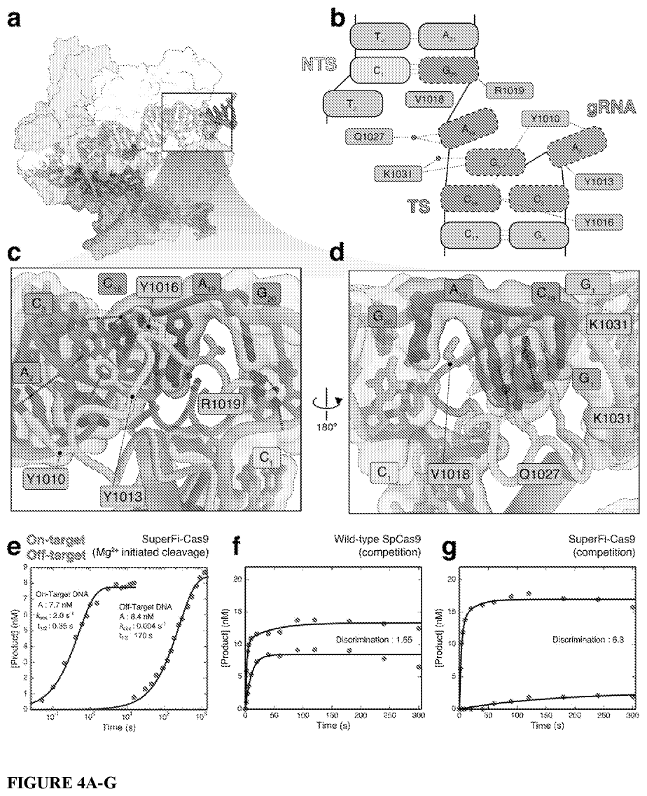

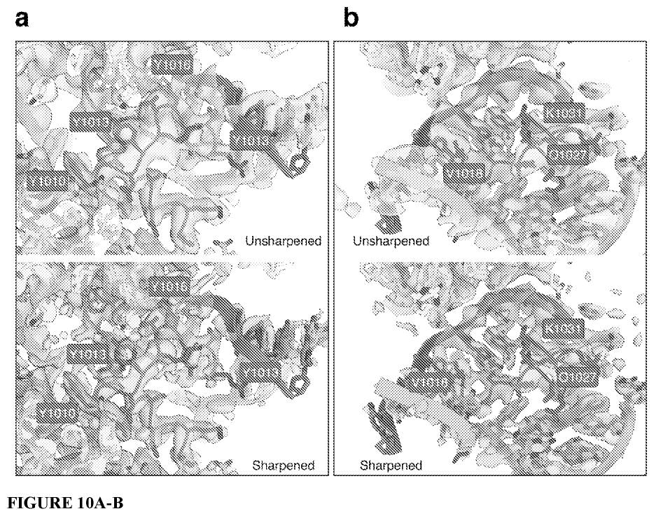

The 18-20 MM contains an unusual duplex conformation at the site of the mismatch. The C: C mismatch at position 18 on the Target Strand, TS (18), is stabilized by stacking interactions with adjacent Watson-Crick base pairs. However, the gRNA is otherwise distorted with gRNA position 2 (gRNA (2)) flipped out by ˜180° so that gRNA (1) then intercalates between TS (19) & TS (20). TS (19) participates in water-mediated hydrogen bonds to Q1027, and TS (20) resumes base-pairing with NTS (FIG. 4, FIG. 10).

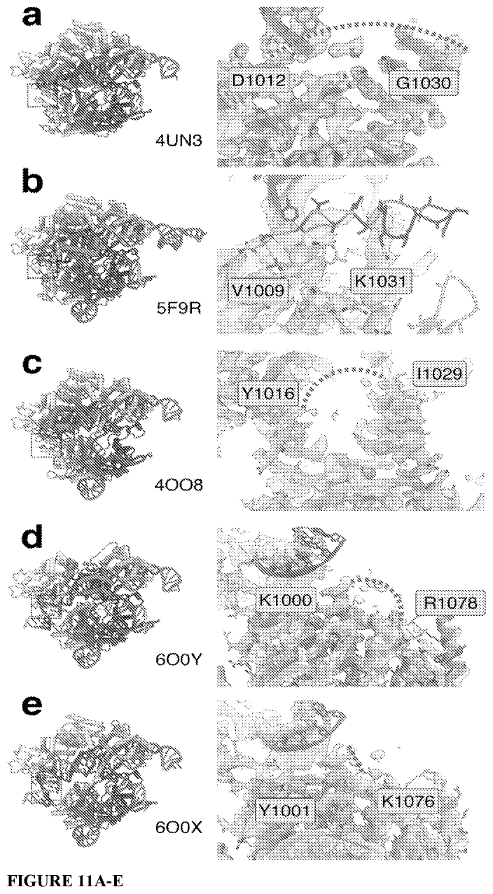

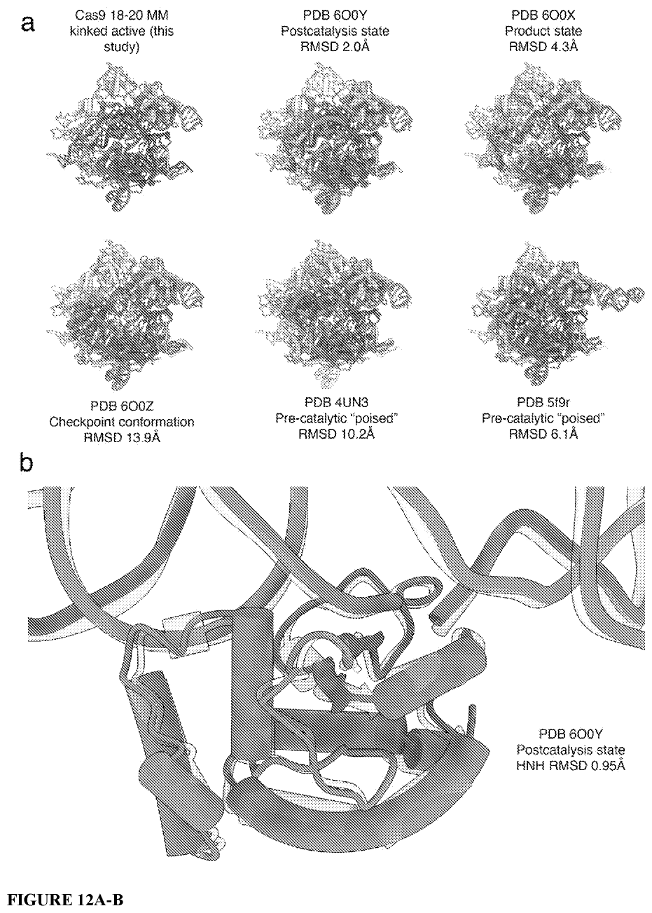

This unusual nucleic acid conformation is stabilized by RuvC and appears to facilitate the binding of this mismatch. The residues within RuvC that contact and stabilize this distorted configuration are absent in previous on-target structures (Anders et al., 2014; Jiang et al., 2016; Nishimasu et al., 2014; Zhu et al., 2019) FIG. 11) despite the overall similarity between this model and a previously-determined active on-target Cas9 (FIG. 12). This indicates that the newly resolved RuvC residues are involved only in mismatch binding rather than on-target activation (FIG. 4). Although this mechanism to accommodate certain mismatches may provide an essential mechanism for bacteria to restrict phage variants, it is counterproductive for use of Cas9 in gene editing.

Previous rationally engineered variants “hyper-accurate Cas9” (HypaCas9: HypaCas9: N692A, M694A, Q695A, and H698A mutations) and “high-fidelity Cas9” (Cas9-HF1: N467A, R661A, Q695A, and Q926A mutations) achieve somewhat higher fidelity at the expense of up to 100-fold reduced efficiency of on-target DNA cleavage (Chen et al., 2017; Kleinstiver et al., 2016; Liu et al., 2020). The mutated residues are mainly located within the REC3 domain and make numerous interactions only with the kinked duplex end. Therefore, by abolishing interactions between REC3 and the PAM-distal duplex, these high-fidelity variants reduce the capacity of Cas9 to stabilize the kinked duplex configuration required for docking of L1 and thereby reduce HNH repositioning and cleavage activity. This data provides a structural explanation for why these high-fidelity Cas9 variants reduce activation of Cas9 (Chen et al., 2017) by off-target substrates, but also reduce on-target Cas9 activity.

To test the role of this loop for mismatch stabilization, a 7-D mutant was designed (where all seven of the stabilizing residues in FIG. 4b are mutated to aspartate) and the effects of this mutant on DNA cleavage were tested. While this 7-D mutant cleaved on-target DNA with a similar rate to WT SpCas9 (2 s⁻¹), it was observed that cleavage of 18-20 MM DNA was 500-fold slower (0.004 s⁻¹) (FIG. 4e). This indicates that this loop is critical for stabilizing the distorted mismatch-induced PAM-distal duplex conformation, thereby allowing the duplex to adopt the kinked conformation that is prerequisite for Cas9 activation. This high-fidelity variant that retains wild type on-target cleavage rates has been named SuperFi-Cas9.

Because enzyme specificity is a kinetic phenomenon that is not determined solely by the rates of the chemical reaction, a direct competition assay was performed, where on-target and off-target (18-20 MM) dsDNA substrates were mixed simultaneously with enzyme and cleavage was monitored over time. While WT Cas9 showed some preference for on-target substrates (a 1.55-fold specificity ratio favoring the on-target over 18-20 MM off-target DNA), SuperFi-Cas9 showed rapid cleavage of on-target while showing minimal cleavage of 18-20 MM DNA (6.3-fold preference for on-target DNA (FIG. 4f & g). The unique ability to discriminate between on- and off-target DNA substrates without compromising DNA cleavage efficiency appears to be unique to SuperFi-Cas9 (Kim et al., 2020).

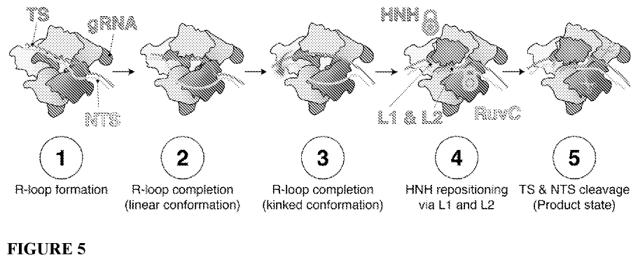

Through kinetics-guided structural determination, a novel gRNA:TS duplex conformational intermediate was identified that precedes Cas9 activation (FIG. 5). Strikingly, it was observed that the well-characterized and widespread off-target cleavage of DNA containing mismatches at the extreme PAM-distal end (positions 18-20 (Chen et al., 2017; Kuscu et al., 2014; Liu et al., 2020; Sternberg et al., 2015; Tsai et al., 2015)) is attributed to a unique mechanism that stabilizes a highly distorted duplex conformation, involving a domain loop in RuvC that penetrates the duplex. Excitingly, this region is missing in previously determined structures of Cas9, showing that it plays a role solely in mismatch tolerance at these positions. The results provide molecular insights into the underlying structural mechanisms that govern off-target effects of Cas9 and provide a molecular blueprint for the design of high fidelity Cas9 variants that reduce off-target DNA cleavage, while retaining efficient cleavage of on-target DNA.

### Materials and Methods

### Protein Expression and Purification

SpCas9 was expressed and purified as described previously (Liu et al., 2020).

### Nucleic Acid Preparation

55-nt DNA duplexes were prepared from PAGE-purified oligonucleotides synthesized by Integrated DNA Technologies. DNA duplexes used in cleavage assays were prepared by mixing 6-FAM or Cy3 labeled target strands with unlabeled non-target strands at a 1:1.15 molar ratio in annealing buffer (10 mM Tris-HCl pH 8, 50 mM NaCl, 1 mM EDTA), heating to 95° C. for 5 minutes, then cooling to room temperature over the course of 1 hour. The sgRNA was purchased from Synthego and annealed in annealing buffer using the same protocol as for the duplex DNA substrates. The sequences of the synthesized oligonucleotides, including the positions of mismatches, are listed in Table 1.

### Kinetics

### Buffer Composition for Kinetic Reactions

Cleavage reactions were performed in 1× cleavage buffer (20 mM Tris-Cl, pH 7.5, 100 mM KCl, 5% glycerol, 1 mM DTT) at 37° C.

### DNA Cleavage Kinetics

The reaction of Cas9 with on- and off-target DNA was performed by preincubating Cas9.gRNA (28 nM active-site concentration of Cas9, 100 nM gRNA) with 10 nM DNA with a 6-FAM label on the target strand in the absence of Mg²⁺. The reaction was initiated by adding Mg²⁺ to 10 mM, then stopped at various times by mixing with 0.3 M EDTA (FIG. 6). Products of the reaction were resolved and quantified using an Applied Biosystems DNA sequencer (ABI 3130xl) (Dangerfield et al., 2021). Data were fit using either a single or double-exponential equations shown below:

Single exponential equation:

Y=A₁e−^{λ}^{₁}^{t}+C Equation (1)
where Y represents concentration of cleavage product, A₁ represents the amplitude, and λ₁ represents the observed decay rate (eigenvalue). The half-life was calculated as t_{1/2}=ln(2)/λ₁.

### Double Exponential Equation:

Y=A₁e^{-λ}^{₁}^{t}+A₂e−^{λ}^{₂}^{t}+C Equation (2)
where Y represents concentration of cleavage product, A₁ represents the amplitude and λ₁ represents the observed rate for the first phase. A₂ represents the amplitude and λ₂ represents the observed rate for the second phase.

### Kinetic Competition Assay

Enzyme specificity is a kinetic phenomenon which is a function of all steps leading up to and including the first largely irreversible step in the pathway and it is common for mutants to introduce a change in specificity determining steps (Johnson, 2019). Therefore, an assay was designed to monitor relative rates of cleavage for on- and off-target DNA when the enzyme was presented with both substrates simultaneously. The competition assay was performed by mixing a solution of 25 nM (active site concentration) Cas9 and 100 nM sgRNA, in the presence of 10 mM Mg²⁺, with 50 nM on-target DNA and 50 nM off-target DNA, where the DNA contained a 5′-6-FAM label or a 5′-Cy3 label on the target or off-target DNA, respectively. Times points were collected by mixing with 0.3 M EDTA and reaction products were resolved and quantified by capillary electrophoresis, as described above. On target cleavage data were fit to a single exponential function and off target cleavage data were fit to a double exponential function. Discrimination was calculated as the ratio of the total amplitude of on target cleavage divided by the amplitude for off target cleavage to derive the relative specificity constants for the on-target DNA compared to the off-target DNA.

### CryoEM Sample Preparation, Data Collection and Processing

Cas9 in complex with various mismatched DNA substrates was frozen at different timepoints, based on kinetic analysis (FIG. 6). A non-productive mismatch complex (15-17 MM, 1h), a slow productive mismatch (12-14) at early (5 min) and late (1h) time points, and a fast productive mismatch (18-20, 1 min) were chosen. MDCC-Cas9 was used for structure determination in order to couple structural analysis with ongoing kinetic studies monitoring changes in fluorescence. It was shown that the kinetics of MDCC-Cas9 were indistinguishable from wild type enzyme (Liu et al., 2020). The cleavage reaction was triggered by mixing 10 μM DNA duplex preincubated with 10 mM MgCl2 and 8 μM MDCC-labelled Cas9: 8 μM gRNA was preincubated with 10 mM MgCl2, in reaction buffer (19 mM Tris-Cl, pH 7.5, 95 mM KCl, 4.75% glycerol, and 5 mM DTT) at a 1:1 ratio. 4 μl of sample was applied to glow discharged holey carbon grids (C-flat 2/2, Protochips Inc.), blotted for 1 s with a blot force of 4 and rapidly plunged into liquid nitrogen-cooled ethane using an FEI Vitrobot MarkIV. Reactions were quenched through vitrification.

Data were collected on an FEI Titan Krios cryo-electron microscope equipped with a K3 Summit direct electron detector (Gatan, Pleasanton, CA). Images were recorded with SerialEM (Mastronarde, 2005) with a pixel size of 1.1 Å for 12-14 MM datasets, and 0.81 Å for 18-20 MM and 15-17 MM datasets, over a defocus range of −1.5 to −2.5 μm. During collection of the 12-14 MM 5 min timepoint dataset, a preferred orientation was observed. To ameliorate this, a second dataset was collected at 30° tilt. Movies were recorded at 13.3 electrons/pixel/second for 6 seconds (80 frames) to give a total dose of 80 electrons/pixel. CTF correction, motion correction and particle picking were performed in real-time using cryoSPARC live. Further data processing was performed cryoSPARC v3.2 (Punjani et al., 2017).

Multiple rounds of 3D classification within cryoSPARC yielded reconstructions of 6 distinct Cas9 complexes at resolutions ranging from 2.7-3.6 Å (Table 3). In order to aid separation of multiple Cas9 conformational states from within the same dataset, 3D variability analysis was performed within CryoSPARC. First and last frames from suitable eigenvector trajectory were then used as references for heterogeneous refinement (i.e. reference-based 3D classification), and particles from resulting classes were refined using non-uniform refinement was used for final reconstructions (Punjani et al., 2020). Active Cas9 (PDB ID 600X) was rigid-body fitted into each map using ChimeraX (Pettersen et al., 2021). Regions of the model not present in a given map were truncated, and flexible fitting was performed using Namdinator (Kidmose et al., 2019). Further modelling was performed using Isolde (Croll, 2018), and the models were ultimately subjected to real-space refinement as implemented in Phenix.

### Materials and Data Availability

The structures of 12-14 MM 5 min, 12-14 MM 60 min linear and 18-20 MM 1 min kinked active have been and their associated atomic coordinates have been deposited into the Electron Microscopy Data Bank (EMDB) and Protein Data Bank (PDB) with accession codes EMD-24833, EMD-24835, EMD-24838 and PDB codes 7S4U, 7S4V and 7S4X, respectively. Maps of 12-14 MM 60 min linear, 15-17 MM 60 min linear and 18-20 1 min linear have been deposited into the Electron Microscopy Data Bank (EMDB) with accession codes EMD-23834. EMD-24836 and EMD-24837. respectively.

### Tables

TABLE 1
List of nucleotide sequences used in Example 1

| Name | Sequence (5′-3′) | Source |
|---|---|---|
| On-target TS | /6-FAM/agc tga cgt ttg tac tcc agc gtc tca tct tta tgc gtc agc aga | IDT |
| | gat ttc tgc t (SEQ ID NO: 2) | |
| On-target NTS | agc aga aat ctc tgc tga cgc ata aag atg aga cgc tgg agt aca aac gtc | IDT |
| | agc t (SEQ ID NO: 3) | |
| 6-8 MM TS | /6-FAM/agc tga cgt ttg tac tcc agc gtc agt tct tta tgc gtc agc aga | IDT |
| | gat ttc tgc t (SEQ ID NO: 4) | |
| 6-8 MM NTS | agc aga aat ctc tgc tga cgc ata aag aac tga cgc tgg agt aca aac gtc | IDT |
| | agc tct cg (SEQ ID NO: 5) | |
| 9-11 MM TS | /6-FAM/agc tga cgt ttg tac tcc agc gtc tca aga tta tgc gtc agc aga | IDT |
| | gat ttc tgc t (SEQ ID NO: 6) | |
| 9-11 MM NTS | agc aga aat ctc tgc tga cgc ata atc ttg aga cgc tgg agt aca aac gtc | IDT |
| | agc tct cg (SEQ ID NO: 7) | |
| 12-14 MM TS | /6-FAM/agc tga cgt ttg tac tcc agc gtc tca tct aat tgc gtc agc aga | IDT |
| | gat ttc tgc t (SEQ ID NO: 8) | |
| 12-14 MM NTS | agc aga aat ctc tgc tga cgc aat tag atg aga cgc tgg agt aca aac gtc | IDT |
| | agc tct cg (SEQ ID NO: 9) | |
| 15-17 MM TS | /6-FAM/agc tga cgt ttg tac tcc agc gtc tca tct tta acg gtc agc aga | IDT |
| | gat ttc tgc t (SEQ ID NO: 10) | |
| 15-17 MM NTS | agc aga aat ctc tgc tga ccg tta aag atg aga cgc tgg agt aca aac gtc | IDT |
| | agc tct cg (SEQ ID NO: 11) | |
| 18-20 MM TS | /6-FAM/agc tga cgt ttg tac tcc agc gtc tca tct tta tgc cag agc aga | IDT |
| | gat ttc tgc t (SEQ ID NO: 12) | |
| 18-20 MM NTS | agc aga aat ctc tgc tct ggc ata aag atg aga cgc tgg agt aca aac gtc | IDT |
| | agc t SEQ ID NO: 13) | |
| sgRNA | GACGCAUAAAGAUGAGACGC + 80-mer SpCas9 scaffold (SEQ ID NO: 14) | Synthego |

TABLE 2
correlation between fraction DNA cleaved and fraction of
cryo-EM particles in linear or kinked duplex conformations.

| | | | % of particles in |
|---|---|---|---|
| | | | linear/kinked |
| Mismatch | Timepoint | % DNA cleaved | conformation |
| 12-14 | 5 min | 9 | 100/0 |
| 12-14 | 60 min | 82 | 42/58 |
| 15-17 | 60 min | 19 | 100/0 |
| 18-20 | 1 min | 63 | 61/39 |

TABLE 3
Cryo-EM data collection, refinement and validation statistics

| | | | | | |
|---|---|---|---|---|---|
| | | 12-14 MM | | | |
| 12-14 MM | | 60 min | | | 18-20 MM |
| 5 min | 12-14 MM | (kinked | 15-17 MM | 18-20 MM | 1 min |
| (linear) | 60 min | pre-active) | 60 min | 1 min | (kinked) |
| (EMD-24833) | (linear) | (EMD-24835) | (linear) | (linear) | (EMD-24838) |
| (PDB 7S4U) | (EMD-24834) | (PDB 7S4V) | (EMD-24836) | (EMD-24837) | (PDB 7S4X) |

Data collection and processing

| Magnification | 22,500 | 22,500 | 22,500 | 29,000 | 29,000 | 29,000 |
|---|---|---|---|---|---|---|
| Voltage (kV) | 300 kV | | | | | |
| Electron exposure (e−/Å²) | 80 | 80 | 80 | 70 | 70 | 70 |
| Defocus range (μm) | −1.5 to −2.5 | | | | | |
| Pixel size (Å) | 1.1 | 1.1 | 1.1 | 0.81 | 0.81 | 0.81 |
| Symmetry imposed | C1 | | | | | |
| Initial particle images (no.) | 1,546,987 | 1,185,683 | 1,185,683 | 1,200,112 | 997,043 | 997,043 |
| Final particle images (no.) | 376,601 | 139,113 | 198,005 | 222,693 | 163,647 | 104,658 |
| Map resolution (Å) | 3.57 | 3.47 | 3.28 | 3.33 | 2.87 | 2.76 |
| FSC threshold | | | | | | |

| | | |
|---|---|---|
| Map resolution range (Å) | 3-7 | 2-6 |

Refinement

| | | | | | | |
|---|---|---|---|---|---|---|
| Initial model used (PDB | 6O0X | N/A | 6O0X | N/A | N/A | 6O0X |
| code) | | | | | | |
| Model resolution (Å) | 3.7 | N/A | 3.4 | N/A | N/A | 3.0 |
| FSC threshold | 0.5 | 0.5 | 0.5 | | | 0.5 |
| Map sharpening B factor | 202.1 | 92.0 | 96.8 | 111.7 | 59.3 | 68.8 |
| (Å²) | | | | | | |

Model composition

| | | | | | | |
|---|---|---|---|---|---|---|
| Non-hydrogen atoms | 11934 | N/A | 12221 | N/A | N/A | 14495 |
| Protein residues | 1092 | | 1122 | | | 1354 |
| Nucleotides | 142 | | 145 | | | 161 |
| Ligands | 0 | | 0 | | | 5 (Mg²⁺) |

Mean B factors (Å²)

| | | | | | | |
|---|---|---|---|---|---|---|
| Protein | 43.8 | N/A | 30.98 | N/A | N/A | 54.68 |
| Nucleotides | 88.68 | | 53.58 | | | 80.66 |

R.m.s. deviations

| | | | | | | |
|---|---|---|---|---|---|---|
| Bond lengths (Å) | 0.004 | N/A | 0.004 | N/A | N/A | 0.006 |
| Bond angles (°) | 0.75 | | 0.555 | | | 0.588 |

Validation

| | | | | | | |
|---|---|---|---|---|---|---|
| MolProbity score | 1.55 | N/A | 1.58 | N/A | N/A | 1.66 |
| Clashscore | 6.11 | | 4.66 | | | 6.97 |
| Poor rotamers (%) | 0 | | 0 | | | 0 |

Ramachandran plot

| | | | | | | |
|---|---|---|---|---|---|---|
| Favored (%) | 96.65 | N/A | 95.05 | N/A | N/A | 95.99 |
| Allowed (%) | 3.35 | | 4.95 | | | 4.01 |
| Disallowed (%) | 0 | | 0 | | | 0 |

TABLE 4
Kinetic parameters determined from Mg²⁺ initiated
cleavage experiments for SuperFi-Cas9.

| | k_{obs} (s⁻¹) | Amplitude (nM) |
|---|---|---|
| On Target | 2.0 ± 0.1 | 7.7 ± 0.1 |
| Off-Target | 0.004 ± 0.0002 | 8.4 ± 0.2 |

### REFERENCES

Aldag, P., Welzel, F., Jakob, L., Schmidbauer, A., Rutkauskas, M., Fettes, F., Grohmann, D., and Seidel, R. (2021). Probing the stability of the SpCas9-DNA complex after cleavage. BioRxiv 2021.08.04.455019.
Anders, C., Niewoehner, O., Duerst, A., and Jinek, M. (2014). Structural basis of PAM-dependent target DNA recognition by the Cas9 endonuclease. Nature 513, 569-573.
Casalino, L., Nierzwicki, Ł., Jinek, M., and Palermo, G. (2020). Catalytic Mechanism of Non-Target DNA Cleavage in CRISPR-Cas9 Revealed by Ab Initio Molecular Dynamics. ACS Catal. 10, 13596-13605.
Chen, J. S., Dagdas, Y. S., Benjamin, P., Welch, M. M., Sousa, A. A., Harrington, L. B., Sternberg, S. H., Joung, J. K., Yildiz, A., and Doudna, J. A. (2017). Enhanced proofreading governs CRISPR-Cas9 targeting accuracy. Nature 550, 407-410.
Cofsky, J. C., Soczek, K. M., Knott, G. J., Nogales, E., and Doudna, J. A. (2021). CRISPR-Cas9 bends and twists DNA to read its sequence. BioRxiv.
Cong. L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science (80-.). 339, 819-823.
Croll, T. I. (2018). ISOLDE: a physically realistic environment for model building into low-resolution electron-density maps. Acta Crystallogr. Sect. D 74, 519-530.
Dagdas, Y. S., Chen, J. S., Sternberg, S. H., Doudna, J. A., and Yildiz, A. (2017). A conformational checkpoint between DNA binding and cleavage by CRISPR-Cas9. Sci. Adv. 1-9.
Doudna, J. A. (2020). The promise and challenge of therapeutic genome editing. Nature 578, 229-236.
Fu, Y., Foden, J. A., Khayter, C., Maeder, M. L., Reyon, D., Joung, J. K., and Sander, J. D. (2013). High-frequency off-target mutagenesis induced by CRISPR-Cas nucleases in human cells. Nat. Biotechnol. 31, 822-826.
Gong. S., Yu, H. H., Johnson, K. A., and Taylor, D. W. (2018). DNA Unwinding Is the Primary Determinant of CRISPR-Cas9 Activity. Cell Rep. 22, 359-371.
Jiang, F., Taylor, D. W., Chen, J. S., Kornfeld, J. E., Zhou, K., Thompson, A. J., Nogales, E., and Doudna, J. A. (2016). Structures of a CRISPR-Cas9 R-loop complex primed for DNA cleavage. Science (80-.). 351, 867-871.
Jinek, M., East, A., Cheng, A., Lin, S., Ma, E., and Doudna, J. (2013). RNA-programmed genome editing in human cells. Elife 2013, 1-9.
Jones, S. K., Hawkins, J. A., Johnson, N. V., Jung, C., Hu, K., Rybarski, J. R., Chen, J. S., Doudna, J. A., Press, W. H., and Finkelstein, I. J. (2021). Massively parallel kinetic profiling of natural and engineered CRISPR nucleases. Nat. Biotechnol. 39, 84-93.
Kellinger, M. W., and Johnson, K. A. (2010). Nucleotide-dependent conformational change governs specificity and analog discrimination by HIV reverse transcriptase. Proc. Natl. Acad. Sci. U.S.A 107, 7734-7739.
Kidmose, R. T., Juhl, J., Nissen, P., Boesen, T., Karlsen, J. L., and Pedersen, B. P. (2019). Namdinator—Automatic molecular dynamics flexible fitting of structural models into cryo-EM and crystallography experimental maps. IUCrJ 6, 526-531.
Kim, D., Luk, K., Wolfe, S. A., and Kim, J. S. (2019). Evaluating and enhancing target specificity of gene-editing nucleases and deaminases. Annu. Rev. Biochem. 88, 191-220.
Kim, N., Kim, H. K., Lee, S., Seo, J. H., Choi, J. W., Park, J., Min, S., Yoon, S., Cho, S. R., and Kim, H. H. (2020)). Prediction of the sequence-specific cleavage activity of Cas9 variants. Nat. Biotechnol. 38, 1328-1336.
Kleinstiver, B. P., Pattanayak, V., Prew, M. S., Tsai, S. Q., Nguyen, N. T., Zheng, Z., and Joung, J. K. (2016). High-fidelity CRISPR-Cas9 nucleases with no detectable genome-wide off-target effects. Nature 529, 490-495.
Kuscu. C., Arslan, S., Singh, R., Thorpe, J., and Adli, M. (2014). Genome-wide analysis reveals characteristics of off-target sites bound by the Cas9 endonuclease. Nat. Biotechnol. 32.677-683.
Liu, M. Sen, Gong, S., Yu, H. H., Jung, K., Johnson, K. A., and Taylor, D. W. (2020). Engineered CRISPR/Cas9 enzymes improve discrimination by slowing DNA cleavage to allow release of off-target DNA. Nat. Commun. 11, 1-13.
Mastronarde, D. N. (2005). Automated electron microscope tomography using robust prediction of specimen movements. J. Struct. Biol. 152, 36-51.
Pettersen, E. F., Goddard, T. D., Huang, C. C., Meng, E. C., Couch, G. S., Croll, T. I., Morris, J. H., and Ferrin, T. E. (2021). UCSF ChimeraX: Structure visualization for researchers, educators, and developers. Protein Sci. 30, 70-82.
Punjani, A., Rubinstein, J. L., Fleet, D. J., and Brubaker, M. A. (2017). CryoSPARC: Algorithms for rapid unsupervised cryo-EM structure determination. Nat. Methods/4, 290-296.
Punjani, A., Zhang, H., and Fleet, D. J. (2020). Non-uniform refinement: adaptive regularization improves single-particle cryo-EM reconstruction. Nat. Methods 17, 1214-1221.
Ran, F. A., Hsu, P. D., Wright, J., Agarwala, V., Scott, D. A., and Zhang, F. (2013). Genome engineering using the CRISPR-Cas9 system. Nat. Protoc. 8, 2281-2308.
Singh, D., Wang, Y., Mallon, J., Yang, O., Fei, J., Poddar, A., Ceylan, D., Bailey, S., and Ha. T. (2018). Mechanisms of improved specificity of engineered Casos revealed by single-molecule FRET analysis. Nat. Struct. Mol. Biol. 25.
Slaymaker. I. M., and Gaudelli, N. M. (2021). Engineering Cas9 for human genome editing. Curr. Opin. Struct. Biol. 69, 86-98.
Slaymaker. I. M., Gao, L., Zetsche, B., Scott. D. A., Yan, W. X., and Zhang. F. (2016). Rationally engineered Cas9 nucleases with improved specificity. Science (80-.). 351, 84-88.
Sternberg. S. H., Lafrance, B., Kaplan, M., and Doudna, J. A. (2015). Conformational control of DNA target cleavage by CRISPR-Cas9. Nature 527, 110-113.
Sun, W., Yang, J., Cheng, Z., Amrani, N., Liu, C., Wang. K., Ibraheim, R., Edraki, A., Huang. X., Wang. M., et al. (2019). Structures of Neisseria meningitidis Cas9 Complexes in Catalytically Poised and Anti-CRISPR-Inhibited States. Mol. Cell 76, 938-952.e5.
Tsai, S. Q., Zheng. Z., Nguyen, N. T., Liebers, M., Topkar, V. V., Thapar. V., Wyvekens. N.,
Khayter, C., Iafrate, A. J., Le, L. P., et al. (2015). GUIDE-seq enables genome-wide profiling of off-target cleavage by CRISPR-Cas nucleases. Nat. Biotechnol. 33, 187-198.
Zhang, Y., Zhang, H., Xu, X., Wang, Y., Chen, W., Wang, Y., Wu, Z., Tang, N., Wang, Y., Zhao, S., et al. (2020). Catalytic-state structure and engineering of Streptococcus thermophilus Cas9. Nat. Catal. 3, 813-823.
Zhu, X., Clarke, R., Puppala, A. K., Chittori, S., Merk, A., Merrill, B. J., Simonovic, M., and Subramaniam, S. (2019). Cryo-EM structures reveal coordinated domain motions that govern DNA cleavage by Cas9. Nat. Struct. Mol. Biol. 26, 679-685.
Johnson, K. A. (2019) Kinetic Analysis for the New Enzymology, KinTek Corporation, Austin
Liu, M. S., Gong, S., Yu, H. H., Jung, K., Johnson, K. A., and Taylor, D. W. (2020) Engineered CRISPR/Cas9 enzymes improve discrimination by slowing DNA cleavage to allow release of off-target DNA. Nat Commun 11, 3576
Liu, M. S., Gong, S., Yu, H. H., Taylor, D. W., and Johnson, K. A. (2019) Kinetic characterization of Cas9 enzymes. Methods Enzymol. 616, 289-311
Gong, S., Yu, H. H., Johnson, K. A., and Taylor, D. W. (2018) DNA Unwinding Is the Primary Determinant of CRISPR-Cas9 Activity. Cell reports 22, 359-371

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. An isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity.

2. The isolated Cas9 variant or fragment thereof of claim 1, wherein the RuvC domain is found within SEQ ID NO: 1.

3. The isolated Cas9 variant or fragment thereof according to claim 1, wherein the at least one mutation is in at least one of residues K1000, Y1001, P1002, K1003, L1004, E1005, S1006, E1007, F1008, V1009, Y1010, G1011, D1012 Y1013, K1014 V1015, Y1016, D1017, V1018, R1019, K1020, M1021, I1022, A1023, K1024, S1025, E1026, Q1027, E1028, 11029, G1030, K1031, A1032, T1033, A1034, K1035, Y1036, F1037, F1038, Y1039, S1040, or N1041 of SEQ ID NO: 1.

4. The isolated Cas9 variant or fragment thereof according to claim 1, wherein the isolated Cas9 variant or fragment thereof has at least one improved property when compared to said parent Cas9 polypeptide.

5. The isolated Cas9 variant or fragment thereof according to claim 4, wherein the isolated Cas9 variant or fragment thereof has improved specificity when compared to said parent Cas9 polypeptide.

6. The isolated Cas9 variant or fragment thereof according to claim 5, wherein the isolated Cas9 variant or fragment thereof does not have greater than a tenfold decrease in specificity of as compared to said parent Cas9 polypeptide.

7. A composition comprising the isolated Cas9 variant or fragment thereof of claim 1.

8. The composition of claim 7, wherein said composition is a ribonucleoprotein complex, wherein said ribonucleoprotein comprises the isolated Cas9 variant or fragment thereof and a gRNA complex.

9. The composition of claim 8, wherein the gRNA complex comprises sgRNA.

10. The composition of claim 8, wherein the gRNA complex comprises tracrRNA and crRNA.

11. The composition of claim 9, wherein said ribonucleoprotein complex is capable of recognizing, binding to, and optionally nicking, unwinding, or cleaving all or part of a target sequence.

12. An expression vector encoding the isolated Cas9 variant or fragment thereof of claim 1.

13. The expression vector of claim 12, wherein the vector further encodes a CRISPR molecule.

14. The expression vector of claim 13, wherein the vector further encodes one or more additional elements necessary to form a ribonucleoprotein complex.

15. A cell encoding the expression vector of claim 12.

16. A method of performing gene editing, the method comprising contacting a target site with an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity.

17-32. (canceled)

33. A method of treating a subject with a disease or disorder which is treatable with gene editing, the method comprising contacting a target site of one or more genes in need of editing within the genome of the subject with an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity, wherein said Cas9 variant or fragment edits one or more genes in a manner which effectively treats said disease or disorder.

34-44. (canceled)

45. A method of modifying an organism to produce a non-naturally occurring product, or a naturally occurring product in a non-naturally occurring amount, the method comprising contacting a target site of one or more genes within the genome of the organism with an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity, wherein said Cas9 edits one or more genes of interest so that the organism produces a non-naturally occurring product, or a naturally occurring product in a non-naturally occurring amount.

46-47. (canceled)

48. A kit comprising an isolated Cas9 variant or a fragment thereof having at least 80% sequence identity to a parent Cas9 polypeptide as set forth in SEQ ID NO: 1, wherein the isolated Cas9 variant or fragment thereof has at least one mutation in RuvC domain, and further wherein said isolated Cas9 variant or fragment has endonuclease activity.

49-51. (canceled)
